(19) United States
(12) Patent Application Publication
(10) Pub. No.: US 2013/0144126 A1
(43) Pub. Date: Jun. 6, 2013

(54) ENDOSCOPE

(71) Applicant: Olympus Corporation, Tokyo (JP)

(72) Inventor: Taro IEDE, Hachioji-shi (JP)

(73) Assignee: OLYMPUS CORPORATION, Tokyo (JP)

(21) Appl. No.: 13/758,167

(22) Filed: Feb. 4, 2013

Related U.S. Application Data
(63) Continuation of application No. PCT/JP2011/071876, filed on Sep. 26, 2011.

(30) Foreign Application Priority Data
Dec. 6, 2010 (JP) 2010-271547

(51) Int. Cl. A61B 1/005; A61B 1/06; A61B 1/12; A61B 1/04; A61B 1/00; A61B 1/015

(52) U.S. Cl. CPC A61B 1/0055; A61B 1/0052; A61B 1/00114; A61B 1/00068; A61B 1/015; A61B 1/00094; A61B 1/12; A61B 1/04; A61B 1/00117; A61B 1/06; A61B 1/00009
USPC 600/139

(57) ABSTRACT

An endoscope has a helical elastic tube constituting a flexible tubular part. The helical elastic tube comprises a dense coil having an initial tension given to at least one part.

### CROSS-REFERENCE TO RELATED APPLICATIONS

This application is a Continuation Application of PCT Application No. PCT/JP2011/071876, filed Sep. 26, 2011 and based upon and claiming the benefit of priority from prior Japanese Patent Application No. 2010-271547, filed Dec. 6, 2010, the entire contents of all of which are incorporated herein by reference.

### BACKGROUND OF THE INVENTION

1. Field of the Invention

The present invention relates to an endoscope having a flexible tubular part.

2. Description of the Related Art

In general, endoscopes have a flexible tubular part. The flexible tubular part is disclosed in, for example, Jpn. Pat. Appln. KOKAI Publication No. H11-285469. This flexible tubular part is composed of a helical tube made of, for example, metal, a mesh tube arranged outside the helical tube and provided on the helical tube, and an envelope arranged outside the mesh tube and provided on the mesh tube. The mesh tube covers the helical be, and the envelope covers the mesh tube. Thus, the flexible tubular part has a three-layer structure.

The flexible tubular part has flexibility, and flexes on receiving, for example, a load. The flexure is proportional to the load. The greater the load, the greater the flexure will be. The load is an external pressure the flexible tubular part receives from the large intestine when the flexible tubular is inserted into the large intestine and abuts on a bent part such as the sigmoid colon of the large intestine.

### BRIEF SUMMARY OF THE INVENTION

According to an aspect of embodiments, an endoscope having a helical elastic tube constituting a flexible tubular part, the helical elastic tube comprising a dense coil having an initial tension given to at least one part.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing an endoscope according to this invention;

FIG. 2 is a diagram showing the three-layer structure of the flexible tubular part;

FIG. 3A is a diagram showing the helical tube (contact coil) of the first embodiment, exerted with an initial tension;

FIG. 3B is a diagram showing a method of measuring the initial tension;

FIG. 3C is a diagram showing a general helical tube formed by winding a thin strap plate helically;

FIG. 3D is a diagram showing the contact coil flexed, from the state shown in FIG. 3A, with a load greater than the initial tension, deformed;

FIG. 4 is a graph showing the load-flexure relationship observed in the contact coil and general helical tube;

FIG. 5A is a graph showing how the initial tension changes at the distal end part side and proximal end part side of the contact coil in a second embodiment;

FIG. 5B is another graph showing how the initial tension changes at the distal end part side and proximal end part side of the contact coil in a second embodiment;

FIG. 6A is a diagram showing a first modification of the contact coil for use in each embodiment; and

FIG. 6B is a diagram showing a second modification of the contact coil for use in each embodiment.

### DETAILED DESCRIPTION OF THE INVENTION

Embodiments of this invention will be described in detail, with reference to the drawings.

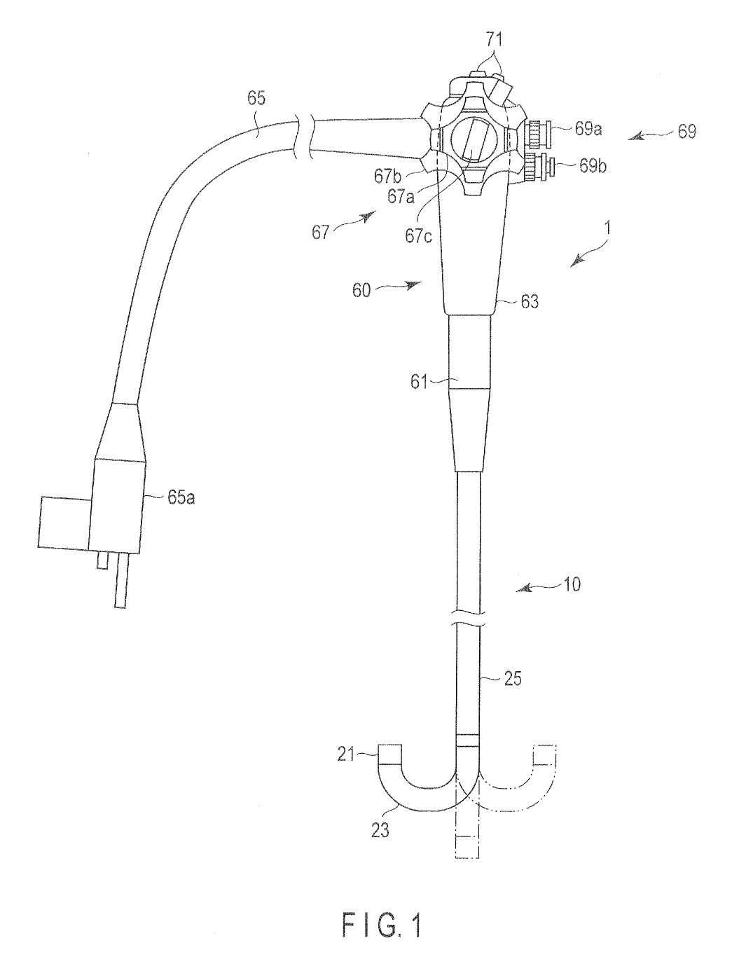

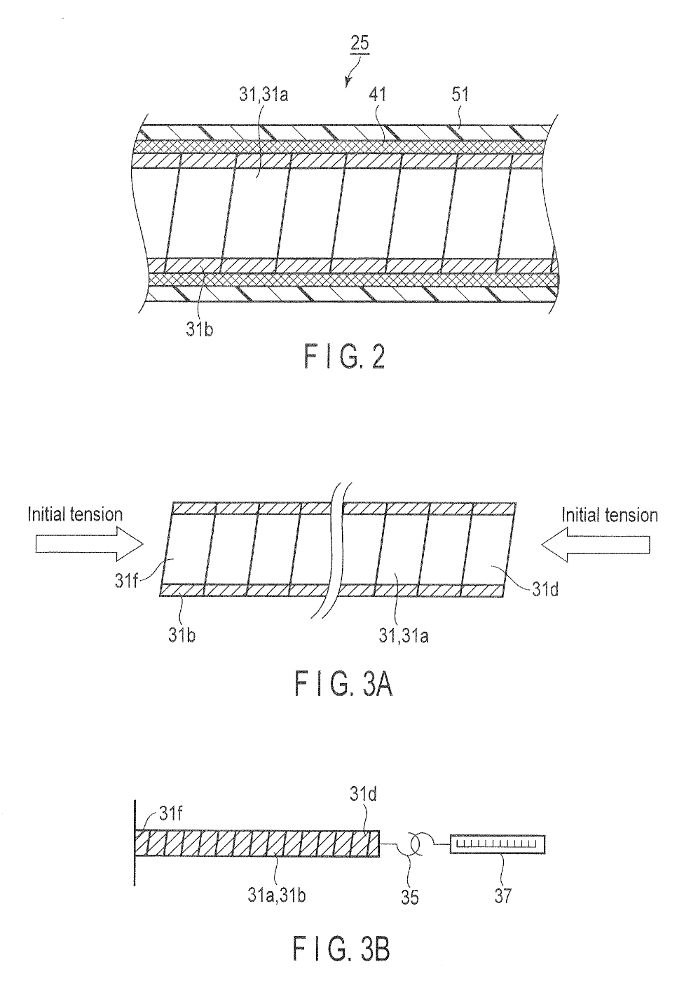

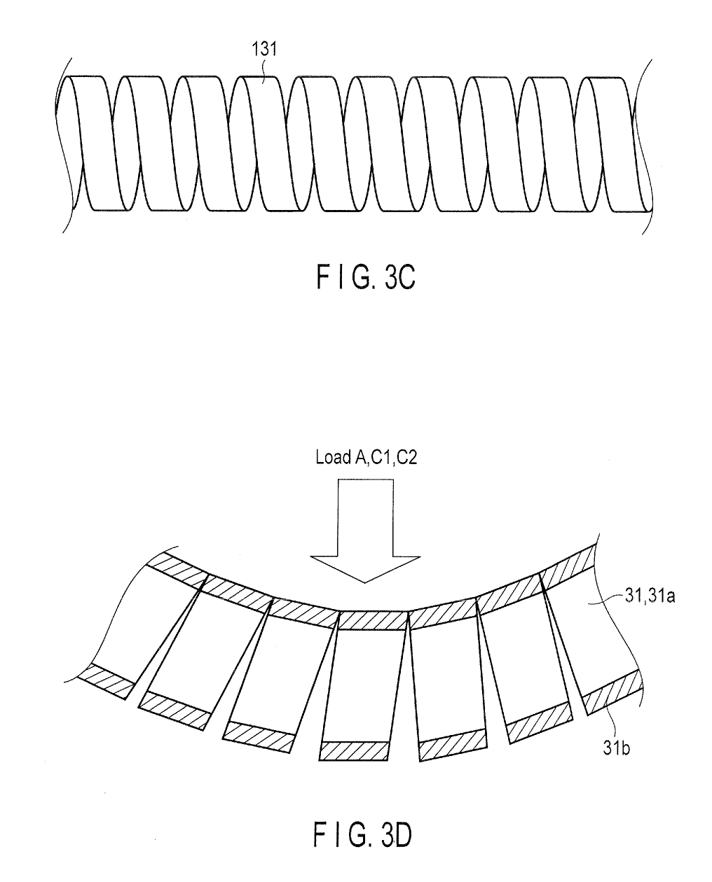

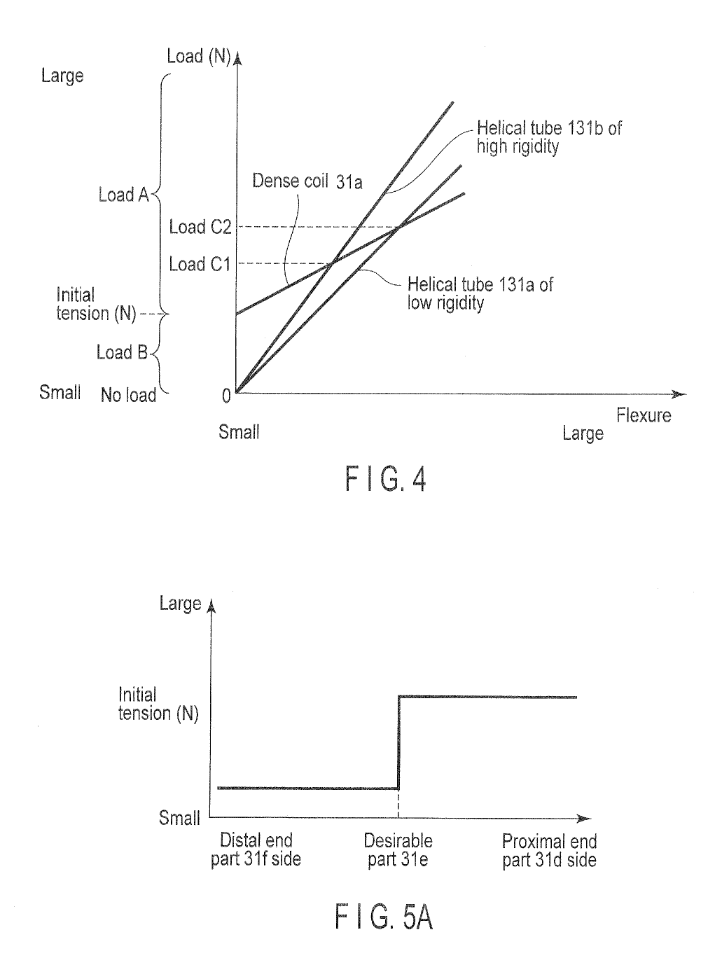

The first embodiment will be described with reference to FIG. 1, FIG. 2, FIG. 3A, FIG. 3B, FIG. 3C, FIG. 3D and FIG. 4.

As shown in FIG. 1, the endoscope 1 has a long thin insertion section 10 which is inserted into a body cavity of the patient and an operation section 60 which is coupled to the proximal end of the insertion section 10 and operates the endoscope 1.

The insertion section 10 has a distal rigid part 21, a bending part 23, and a flexible tubular part 25, which are arranged in the order mentioned, from a distal end part side of the insertion section 10 toward a proximal end part side of the insertion section 10. A proximal end part of the distal rigid part 21 is coupled to a distal end part of the bending part 23. A proximal end part of the bending part 23 is coupled to a distal end part of the flexible tubular part 25.

The distal rigid part 21 is the distal end part of the insertion section 10 and is hard enough not to be bent.

The bending part 23 can be bent to any desired direction, to the left or the right and upwards or downward, as a bending operating unit 67 is operated as will be described later. As the bending part 23 is bent, the distal rigid part 21 changes in position and orientation. As a result, the object to observe will be caught in the observation view field and will be illuminated with illumination light.

The flexible tubular part 25 has desirable flexibility. The flexible tubular part 25 can therefore be bent with an external force applied to it. The flexible tubular part 25 is a tubular member extending from the main part 61 of the operation section 60. The structure of the flexible tubular part 25 will be described later.

The operation section 60 has a main part 61 from which the flexible tube portion 25 is extended, a grip part 63 that is coupled to a proximal end part of the main part 61 and gripped by an operator of the endoscope 1, and a universal cord 65 is connected to the grip portion 63.

The grip part 63 has a bending operating unit 67 configured to bend the bending part 23. The bending operating unit 67 has a left-right bend no knob 67a that bends the bending part 23 in left and right directions, an up-down bending knob 67b that bends the bending part 23 in upward and downward directions, and a position setting knob 67c sets a position of the bent bending part 23.

The grip part 63 further has a switch unit 69. The switch unit 69 has a suction switch 69a and an air/water supply switch 69b. The switch unit 69 may be operated by the operator gripping the grip part 63. The suction switch 69a is operated to draw mucus or fluid through a suction channel (not shown) from the suction port (not shown) provided at the distal rigid part 21. The air/water supply switch 69b is operated to supply fluid through the air/water channel (not shown) of the distal rigid part 21, to secure, at the distal rigid part 21, an observation view field for a photographing unit (not shown). The fluid includes water and air.

The grip part 63 has various buttons 71, which may be pushed to perform endoscope photography.

The universal cord 65 has a connection part 65a, which may be connected to a video processor (not shown) or a light source device (not shown).

With reference to FIG. 1 and FIG. 2, the structure of the flexible tubular part 25 will be described.

The flexible tubular part 25 is a hollow member. More specifically, the flexible tubular part 25 has, for example, a helical tube 31, a mesh tube 41 arranged outside the helical tube 31 and provided on the helical tube 31, and an envelope 51 arranged outside the mesh tube 41 and provided on the mesh tube 41 as shown in FIG. 2. The mesh tube 41 covers the helical tube 31, and the envelope 51 covers the mesh tube 41.

The flexible tubular part 25 is thus composed of the helical tube 31, mesh tube 41 and envelope 51. The flexible tubular part 25 therefore has a three-layer structure. The diameter of the flexible tubular part 25 is, for example, 12 mm.

The helical tube 31 of this embodiment is a helical tubular member having an elastic force. As shown in FIG. 3A and FIG. 4, this elastic tubular member is composed of a dense coil 31a given an initial tension. Since, the helical tube 31 has an elastic force, the dense coil 31a is provided in the form of a dense coil spring. The dense coil 31a is formed of an elemental wire 31b wound helically.

The initial tension is force acting in such a direction that the elemental wire 31b of the dense coil 31a closely contact one another in the lengthwise direction of the dense coil 31a. In other words, the initial tension is the force that keeps the dense coil 31a straight, not flexing at all, even if the dense coil 31a receives an external force while exerted with no load. Hence, when the dense coil 31a receives an external force, while receiving no load, the initial tension keeps the elemental wire 31b remain closely contacting one another in the lengthwise direction, therefore the dense coil 31a would not flex by the initial tension.

The initial tension is applied to the dense coil 31a from a distal end part 31f side of the dense coil 31a and a proximal end part 31d side of the dense coil 31a toward a middle part of the dense coil 31a in the lengthwise direction thereof, as shown in FIG. 3A, as the dense coil 31a is being formed. The initial tense is uniformly applied in the lengthwise direction of the dense coil 31a, for example, from the distal end part 31f to the proximal end part 31d. At this point, the initial tension A is, for example, 0 N<A≦25 N. The initial tension on the dense coil 31a can be adjusted by the direction in which the elemental wire 31b is helically wound.

In order to measure the initial tension, a hook part 35 is formed at the proximal end part 31d of the dense coil 31a as shown in FIG. 3B. The hook part 35 may catch a measuring device 37 such as a digital force gauge. The distal end part 311 of the dense coil 31a is secured, and the measuring device 37, which is caught by the hook part 35, is pulled in the lengthwise direction of the dense coil 31a. The measuring device 37 measures the load applied when the dense coil 31a is pulled (or when the elemental wire 31b are separated from one another). The load thus measured is the initial tension.

As shown in FIG. 3C, a general helical tube 131 is formed by a thin strap made of, for example, stainless steel and wound helically. The helical tube 131 has an almost circular cross section. The helical tube 131 is, for example, a metal helical tube having a small wall thickness.

The helical tube 131 flexes when a load is applied to helical tube 131 in a diameter direction of the helical tube 131. At this point, the flexure (deforming) is proportional to the load. The greater the load, the greater the flexure will be, greater than zero. At the same load, the lower the rigidity of the helical tube 131, the greater the flexure will be. In other words, as shown in FIG. 4, flexure of a helical tube 131a having a low rigidity is greater than flexure of a helical tube 131b having a high rigidity, at the same load.

As seen from FIG. 3D and FIG. 4, the dense coil 31a flexes in accordance with its spring constant if it receives a load greater than the initial tension (hereinafter referred to load A). This load is an external pressure the flexible tubular part 25 receives from the large intestine when the flexible tubular part 25 is inserted into the large intestine and abuts on a bent part such as the sigmoid colon of the large intestine.

In this embodiment, the dense coil 31a has a smaller spring constant than the rigidity of the helical tube 131. The dense coil 31a therefore flexes in accordance with the spring constant.

How the dense coil 31a and the helical tube 131 flex will be explained in detail.

As described above and as shown in FIG. 3A and FIG. 4, the dense coil 31a does not flex by the initial tension, while receiving no load. As shown in FIG. 3A and FIG. 4, the dense coil 31a does not flex even if a load smaller than the initial tension (hereinafter referred to as “load B”) is applied to dense coil 31a in a diameter direction of the dense coil 31a. This is because the elemental wire 31b closely contact one another by virtue of the initial tension. That is, the flexure is zero in this case. Thus, the dense coil 31a remains almost straight when it receives no load or load B.

As shown in FIG. 3A and FIG. 4, the elemental wire 31b separate one from another when load A is applied to the dense coil 31a in the diameter direction of the dense coil 31a. As a result, the dense coil 31a flexes for the first time. That is, the flexure become greater than zero. In other words, the dense coil 31a will not flex if load A is not applied to the dense coil 31a.

When load A is applied to the dense coil 31a, the dense coil 31a flexes as shown in FIG. 4 in proportion to its spring constant lower than the rigidity of the helical tube 131.

If specific load (hereinafter referred to as “load C1” and “load C2”) in load A is applied, the dense coil 31a will flex more than the helical tube 131 does as shown in FIG. 3C at the same load. Note that load C2 is greater than load C1.

If the same load greater than load C1 is applied to the dense coil 31a and to the helical tube 131b, the dense coil 31a will flex more than the helical tube 131b. In other words, if a load greater than load C1 is applied to the dense coil 31a and to the helical tube 131b, and if the dense coil 31a and the helical coil 131b flex to the same degree, the load applied to the dense coil 31a is smaller than the load applied to the helical tube 131b.

If the same load greater than load C2 is applied to the dense coil 31a and to the helical tube 131a, the dense coil 31a will flex more than the helical tube 131a. In other words, if a load greater than load C2 is applied to the dense coil 31a and to the helical tube 131a, and if the dense coil 31a and the helical tube 131a flex to the same degree, the load applied to the dense coil 31a is smaller than the load applied to the helical tube 131a.

In this embodiment, if a load greater than the initial tension and smaller than load C2 is applied to the dense coil 31a, the operation force applied at hand of the operator will be transmitted to the distal end part 31f side of the dense coil 31a. The dense coil 31a therefore flexes a little, but much enough to facilitate the insertion of the flexible tubular part 25 into the body cavity.

How the dense coil 31a flexes has been explained. The flexible tubular part 25, which comprises the dense coil 31a, flexes in the same way as the dense coil 31a does as has been explained.

The dense coil 31a is made of metal such as SUS304. As shown in FIG. 2 and FIG. 3A, the elemental wire 31b of the dense coil 31a has a cross section shaped like a rectangle. The dense coil 31a has a diameter of, for example, 10 mm, and the elemental wire 31b has a thickness of, for example, 0.3 mm.

The mesh tube 41 has been made by knitting a plurality of filaments made of, for example, stainless steel, forming a tubular net having a cross section that is almost circular. In the mesh tube 41, the filaments extend orthogonal, one to another, forming lattices.

The envelope 51 is shaped like a tube having a circular cross section, is made of flexible resin such as rubber, and covers the outer circumferential surface of the mesh tube 41.

The method of operating this embodiment will be an explained.

The helical tube 31 comprises the dense coil 31a applied with the initial tension. The flexible tubular part 25 has the helical tube 31 so constructed.

Therefore, the flexible tubular part 25 keeps extending straight not flex as seen from FIG. 4 even if the flexible tubular part 25 receives a load smaller than the initial tension, which is load B, while the flexible tubular part 25 inserted into the body cavity. The flexure is thus zero. The operation force applied at a hand of the operator the proximal end part is transmitted to the distal end part side of the flexible tubular part 25 (i.e., distal end part 31f side of the helical tube 31), facilitating the insertion of the flexible tubular part 25 into the body cavity. The flexible tubular part 25 can remain straight not flex, though receiving load B, and is inserted into the body cavity.

Even if a load greater than initial tension and smaller than load C1 is applied to the flexible tubular part 25, the flexible tubular part 25 flexes less than the flexible tubular part having the helical tube 131. The operation force applied at the hand of the operator is more readily transmitted to the distal end part 31f side of the helical tube 31 than in the flexible tubular part having the helical tube 131. The flexible tubular part 25 can therefore be easily inserted into the body cavity.

The flexible tubular part 25 may be inserted into the body cavity and may flex with a load greater than the initial tension and smaller than load C1. In this case, if a load greater than load C1 (for example, load C2) is applied to the flexible tubular part 25, the flexible tubular part 25 already flexing will more flex, than the flexible tubular part having the helical tube 131b as shown in FIG. 4.

The flexible tubular part 25 may be inserted into the body cavity and may flex with a load greater than the initial tension and smaller than load C2. In this case, if a load greater than load C2 is applied to the flexible tubular part 25, the flexible tubular part 25 already flexing will more flex than the flexible tubular part having the helical tube 131a as shown in FIG. 4.

Thus, if the flexible tubular part 25 already flexing further flexes in the body cavity, on receiving a load greater than load C2, the flexible tubular part which has the dense coil 31a, will not apply a large tension to the large intestine even if it abuts on a bending part of the large intestine, never imposing a burden on the patient. In this case, the flexible tubular part 25, which has the dense coil 31a, mere flexes than the flexible tubular part having the helical tube 131. In this case, at the same flexure, load for flexible of the flexible tubular part 25, which has the dense coil 31a, is smaller than load for flexible of the flexible tubular part having the helical tube 131. The flexible tubular part 25 is easy to manipulate.

In this embodiment, the helical tube 31 comprises the dense coil 31a given the initial tension. Therefore, the flexure of the flexible tubular part 25 is zero or very small even if a load is applied to the flexible tubular part 25 extending straight. The flexible tubular part 25 already flexing will more flex if a load is further applied to it.

In this embodiment, the flexible tubular part 25 can be easily inserted into the body cavity, either while remaining straight or while flexing a little. Hence, in this embodiment, the operation force applied at a hand of the operator can be more reliably and easily transmitted to the distal end part side of the flexible tubular part 25. The flexible tubular part 25 can therefore be easily inserted into the body cavity.

In this embodiment, in order to flex greatly, the flexible tubular part 25 need not abut on a bending part of the large intestine, do not apply a large tension to the intestine, do not impose a burden on the patient in the body cavity. In this embodiment, the flexible tubular part 25 need not abut on a bending part of the large intestine, do not apply a large tension to the intestine, do not impose a burden on the patient, the flexible tubular part 25 can flex greatly (to increase the flexure) and can flex with a small load. Moreover, the flexible tubular part 25 of this embodiment is easy to manipulate.

In this embodiment, the initial tension is given to the dense coil 31a in the process of forming the dense coil 31a. That is, the initial tension is not given to the dense coil 31a after the dense coil 31a has been formed. This helps to save time and labor in the manufacturer of the dense coil 31a and the flexible tubular part 25.

The flexible tubular part 25 of the embodiment is composed of the helical tube 31 (dense coil 31a), mesh tube 41 and envelope 51. The flexible tubular part 25 therefore has a three-layer structure. The flexible tubular part 25 is not limited to this structure, nevertheless. It is sufficient for the flexible tubular part 25 to have at least the dense coil 31a given an initial tension in its entirety.

In this embodiment, the initial tension is given to the entire dense coil 31a. Instead, the initial tension may be given to at least one part of the dense coil 31a, and the flexible tubular part 25 may have a helical tube 31 (dense coil 31a) at least one part of which is given an initial tension.

In this embodiment, the initial tension is continuously applied to the entire helical tube 31, from the distal end part 31f to proximal end part 31d thereof. Instead, for example, initial tensions given to the distal end part 31f and the proximal end part 31d, and do not give between the distal end part 31f and the proximal end part 31d. That is, the initial tension may be applied unevenly over the helical tube 31. In this case, the initial tensions is, for example, almost the same value.

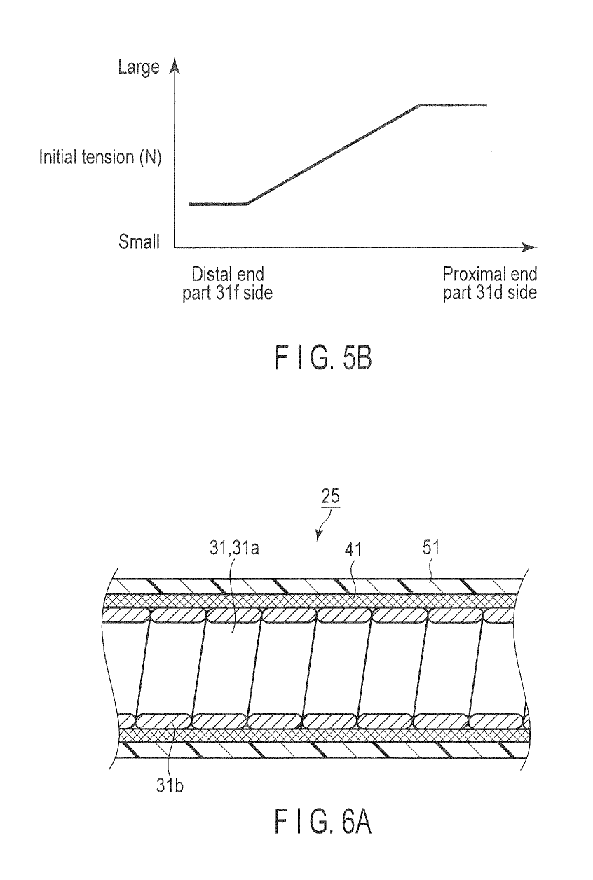

The second embodiment of this invention will be described with reference to FIG. 5A and FIG. 5B. In this embodiment, the initial tension is applied to the dense coil 31a, but not uniformly in the lengthwise direction of the dense coil 31a. For example, the initial tension given to the proximal end part 31d side of the dense coil 31a is greater than the initial tension given to the distal end part 31f side. In this case, as shown in FIG. 5A, the initial tension is small from the distal end part 31f side to a desirable part 31e close to the proximal end part 310 side of the dense coil 31a, and is large from the desirable part 31e to the proximal end part 310. Alternatively, as shown in FIG. 5B, the initial tension may gradually increase from the distal end part 31f side toward the proximal end part 31d side.

Therefore, the distal end part 31f side is a soft part, having a small rigidity. The proximal end part 310 side is a hard part, having a large rigidity.

Since the distal end part 31f side is a soft part in this embodiment, it would not apply a large tension to the intestine even if distal end part 31f side abuts on a bending part of the large intestine, and can be inserted along the intestine. Therefore, the distal end part 31f side can easily be inserted into a body cavity, reducing a burden, if any, imposed on the patient.

In this embodiment, the proximal end part 31d side is formed as a hard part, and the flexible tubular part 25 is therefore prevented from easily flexing even if the operation force applied at the hand of the operator is applied to the flexible tubular part 25, or the operator exerts a force (load) on the flexible tubular part 25. The operation force applied at the proximal end can therefore be easily transmitted to the distal end part 31f side. Hence, the flexible tubular part 25 can be easily inserted into a body cavity.

This embodiment, can attain the above-mentioned advantage if the initial tension is given to only the proximal end part 31d side, not to the distal end part 31f at all. In this case, the initial tension applied to the proximal end part 31d side may be uniform over the entire proximal end part 31d side or may gradually increases toward the proximal end of the proximal end part 31d.

As specified above, the initial tension may be applied unevenly. If so, the initial tensions given to the distal end part 31f side is smaller than the initial tensions given to the proximal end part 31d side. Further, as described above, the initial tension given to the distal end part 31f side gradually increases toward the proximal end part 31d, and the initial tension given to the proximal end part 31d side gradually increases toward the proximal end part 31d. The largest initial tension applied to the distal end part 31f side at this point is, for example, smaller than the smallest initial tension applied to the proximal end part 31d side.

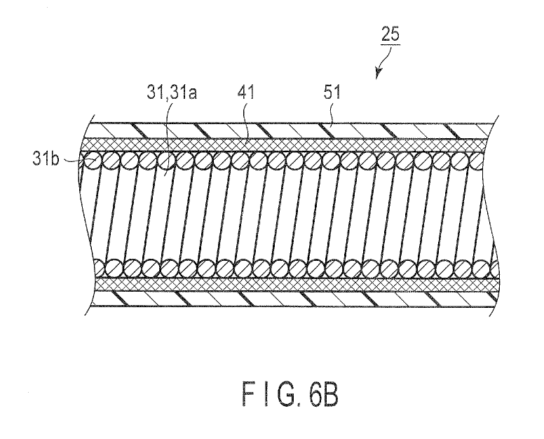

As the first modification of each embodiment described above, the elemental wire 31b of the dense coil 31a may have an oblong cross section as shown in FIG. 6A. Since the elemental wire 31b has an oblong cross section, it can be wound at a more obtuse angle than in the case it has a rectangular cross section. Therefore, the dense coil 31a can acquire a greater initial tension in this modification. Also in this modification, wherein the elemental wire 31b has an oblong cross section, the elemental wire 31b contact one another at points, reducing the area in which any two adjacent turns contact. As a result, the friction between the elemental wire 31b decreases, enabling the dense coil 31a to flex smoothly.

As the second modification of each embodiment described above, the elemental wire 31b of the dense coil 31a may have a circular cross section as shown in FIG. 6B. Since this cross section has no edges, any adjacent two elemental wire 31b would not ride onto each other. As a result, the curvature of the flexible tubular part 25 can be reduced.

In view of the above, it is sufficient for the elemental wire 31b of the dense coil 31a to have at least one of, for example, a rectangular cross section, an oblong cross section and a circular cross section.

The endoscope 1 can be used for medical purpose and for industrial purpose.

This invention is not limited to the embodiments described above. The components of the invention may be modified in reducing the invention to practice, without departing from the scope of the invention. Further, the components of any embodiments described above may be combined, if necessary, in appropriate ways to make various inventions.

What is claimed is:

1. An endoscope having a helical elastic tube constituting a flexible tubular part,
the helical elastic tube comprising a dense coil having an initial tension given to at least one part.

2. The endoscope according to claim 1,
wherein the initial tension given to a proximal part side of the dense coil is greater than the initial tension given to a distal part side of the dense coil.

3. The endoscope according to claim 2,
wherein the dense coil is formed of an elemental wire having at least one of a rectangular cross section, a oblong cross section and a circular cross section.
